(19) United States
(12) Patent Application Publication
(10) Pub. No.: US 2026/0250614 A1
(43) Pub. Date: Aug. 27, 2026

(54) Rapid 3D-Bioprinting of Microfluidic Vascular Tissue Models

(71) Applicant: University of Cincinnati, Cincinnati, OH (US)

(72) Inventor: Riccardo Barrile, Crescent Springs, KY (US)

(73) Assignee: University of Cincinnati, Cincinnati, OH (US)

(21) Appl. No.: 18/853,182

(22) PCT Filed: Apr. 3, 2023
(86) PCT No.: PCT/US2023/017259

§ 371 Date: Oct. 1, 2024

Related U.S. Application Data
(60) Provisional application No. 63/326,569, filed on Apr. 1, 2022.

(51) Int. Cl. C12M 3/00; B33Y 80/00; C12M 1/00; C12M 1/12; C12M 3/06

(52) U.S. Cl. CPC C12M 21/08; B33Y 80/00; C12M 23/16; C12M 25/14; C12M 29/00

(57) ABSTRACT

A novel vascular tissue model is disclosed. The model uses a rigid 3D printed scaffold. This scaffold includes one or more scaffold microfluidic channels, two or more inlets, and a central chamber. The central chamber contains a hydrogel, and the hydrogel includes a hydrogel microfluidic channel.

### CROSS-REFERENCE TO RELATED APPLICATIONS

This application is a continuation of PCT Application No. PCT/US23/17259 filed Apr. 3, 2023, which claims the benefit of the filing date of U.S. Provisional Application No. 63/326,569, filed on Apr. 1, 2022, the disclosures of which are incorporated by reference herein in their entirety.

### TECHNICAL FIELD

Aspects of the present invention relate generally to vascular tissue models

### BACKGROUND OF THE INVENTION

This section is intended to introduce the reader to various aspects of art that may be related to various aspects of the present invention, which are described and/or claimed below. This discussion is believed to be helpful in providing the reader with background information to facilitate a better understanding of various aspects of the present invention. Accordingly, it should be understood that these statements are to be read in this light, and not as admissions of prior art.

The discrepancy between results obtained in preclinical studies, which are typically derived from animal experimentation, and clinical outcomes is the root cause of drug failure across diverse disease areas. The deteriorating attrition rate, which currently sees less than one in ten new drugs obtain approval, highlights the fact that animal models are simply not good predictors of the human biology. Therefore, there is an urgent need to develop more predictive and reliable human-relevant models that are able to faithfully recapitulate the physiological microenvironment of human tissues and organs to provide better alternatives to animal testing.

Recent advancements in bioengineering have led to the development of complex cell culture models, frequently referred to as Microphysiological systems (MPSs) or Organs-on-Chips (OOCs); these microfluidic cell culture devices are composed of optically clear plastic, glass or flexible polymers and contain perfused hollow microchannels populated by living cells. Such microfluidic devices can be used to create “tissue chips” with channels lined by multiple cell types to combine, for example, tissue-specific parenchymal and vascular microchannels, thereby recreating tissue-tissue interfaces that are crucial for reconstituting organ-level structures and functions.

Previous MPSs have been created using polydimethylsiloxane (PDMS) scaffolds along with a porous membrane. This method has numerous disadvantages. It can take weeks using this scaffold to create one MPS. These systems cannot be used for drug testing and assessment of soluble cell metabolites, because small molecules are absorbed by the system. Intercellular interactions are also limited because the membrane results in asymmetrical formation of the tissue-tissue interface. Despite this, the use of PDMS remains the most prominent across nearly all existing MPSs.

One area where MPS could be particularly useful is the study of Glioblastoma multiforme (GBM). GBM is the most common primary adult malignant brain tumor and carries a poor prognosis. Treatment of GBM in the newly diagnosed setting involves surgical resection, followed by chemoradiation (radiation+temozolomide/TMZ). Unfortunately, therapies against this aggressive cancer have a high rate of recurrence due to tumor heterogeneity and the survival of drug-resistant glioblastoma stem cells (GSCs), which repopulate the brain of treated patients. In addition, the protective function of the blood-brain barrier (BBB) can also add to limited treatment options. Molecular mechanisms underpinning drug resistance and tumor regeneration in the brains of patients after therapy are still poorly understood. However, a growing body of evidence indicates the self-renewal potential of GBM is limited to a few stem cells that possess a drug resistance mechanism and reside within distinct regions (niches) of the brain tissue, including perivascular and hypoxic niches. Traditional animal models and simple cell-monoculture systems do not offer adequate translational solutions for identifying the human-relevant mechanism of drug resistance that the tissue microenvironment can mediate.

The emerging organ-on-chip technology offers unique opportunities for dissecting the intercellular communication occurring within GBM and surrounding microenvironment and testing of drug compounds for the purpose of developing novel and personalized therapeutic strategies. Unfortunately, previous models suffered from a few drawbacks associated with conventional microfabrication methods and materials that represents a major obstacle to the development of a predictive GBM-on-chip model suitable for drug testing. PDMS is the most widely used material for fabrication of organ-on-chip because it is inexpensive and has prototyping-friendly properties. However, PDMS exhibits variable and time-dependent absorption of small, hydrophobic molecules, making it possibly unsuitable for drug testing of small molecules, which account for the largest class of therapeutics targeting the central nervous system. An organ-on-chip model of GBM that is compatible with testing of small molecule drug-candidates could significantly enhance the development of novel therapeutics as well as research to elucidate the mechanisms of brain cancer progression and drug resistance.

### SUMMARY OF THE INVENTION

Certain exemplary aspects of the invention are set forth below. It should be understood that these aspects are presented merely to provide the reader with a brief summary of certain forms the invention might take and that these aspects are not intended to limit the scope of the invention. Indeed, the invention may encompass a variety of aspects that may not be explicitly set forth below.

The present invention discloses a novel vascular tissue model. The model uses a rigid 3D printed scaffold. This scaffold includes one or more scaffold microfluidic channels, two or more inlets; and a central chamber. The central chamber contains a hydrogel, and the hydrogel includes a hydrogel microfluidic channel. In one embodiment, the hydrogel microfluidic channel connects to one or more of the scaffold microfluidic channels. In another embodiment, the inlets are capable of connecting to one or more pumps. In one embodiment, the scaffold has an inner surface and the inner surface comprises one or more hydrogel anchoring structures.

In another embodiment, the rigid 3D printed scaffold is created using stereolithography. In one embodiment, the microfluidic scaffold comprises a transparent resin, and further, wherein the microfluidic scaffold is biocompatible with biological material that may be used in the vascular tissue model. In another embodiment, the microfluidic scaffold is surface functionalized.

In one embodiment, the hydrogel is created using three-dimensional bioprinting. In another embodiment, the hydrogel microfluidic channel has a circular cross section. In one embodiment, the hydrogel comprises a material selected from the group consisting of fibrin, collagen, matrigel, alginate, gelatin, synthetic polymers, and tissue-specific extracellular matrix. In another embodiment, the hydrogel comprises stromal cells, brain glioma cells or combinations thereof. In one embodiment, the hydrogel microfluidic channel contains human endothelial cells. In another embodiment, the vascular tissue model is of the human blood-brain barrier.

The present invention also discloses a method of modeling a vascular tissue system. The method involves inserting a culture comprising cancer cells in the hydrogel microfluidic channel of the vascular tissue model described above. The vascular tissue model is connected to one or more pumps. Next, flowing medium through the vascular tissue model, including the hydrogel microfluidic channel. Data is collected regarding the culture in the hydrogel microfluidic channel. In one embodiment, the rigid 3D printed scaffold is created using stereolithography. In another embodiment, the hydrogel is created using three-dimensional bioprinting. In one embodiment, the culture is a co-culture of human endothelial cells with cancer cells. In another embodiment, the cancer cells are brain glioma cells. In one embodiment, the culture comprises stromal cells and brain glioma cells. In another embodiment, the stromal cells are endothelial cells, astroglia cells or combinations thereof.

### BRIEF DESCRIPTION OF THE DRAWINGS

The accompanying drawings, which are incorporated in and constitute a part of this specification, illustrate embodiments of the invention and, together with the general description of the invention given above, and the detailed description given below, serve to explain the principles of the invention. Similar reference numerals are used to indicate similar features throughout the various figures of the drawings.

FIG. 1A is a schematic of a device according to the present invention.

FIG. 1B is a schematic of a device according to the present invention.

FIG. 2A is a schematic of a device according to the present invention.

FIG. 2B is a schematic of a device according to the present invention.

FIG. 3 is a schematic of a device according to the present invention.

FIG. 4A is an image of an industrial 3D printer.

FIG. 4B is an image an assortment of printed scaffolds according to the present invention.

FIG. 4C is an image of a 3D bioprinter.

FIG. 4D is an image of a bioprinter printing a microfluidic vascular channel according to the present invention.

FIG. 4E is an image of photo of a device according to the present invention.

FIG. 5 is a schematic showing the key steps of one embodiment of the approach of the present invention.

FIG. 6A is an illustration of cancer cells with a branched morphology.

FIG. 6B is an illustration of cancer cells with a roundish morphology and endothelial cells.

FIG. 6C is a graph showing a cell shape index.

FIG. 6D is a graph showing the number of round cancer cells for various cultures.

### DEFINITIONS

The present disclosure may be understood more readily by reference to the following detailed description of the embodiments taken in connection with the accompanying drawing figures, which form a part of this disclosure. It is to be understood that this application is not limited to the specific devices, methods, conditions or parameters described and/or shown herein, and that the terminology used herein is for the purpose of describing particular embodiments by way of example only and is not intended to be limiting. Also, in some embodiments, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

As used herein, the term “about,” when referring to a value or to an amount of mass, weight, time, volume, pH, size, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, “rigid” means somewhat inflexible and stiff. “Rigid” includes materials that are not completely inflexible or immovable or unbendable.

As used herein, “3D printed” means a structure formed using a 3D printing technique.

As used herein, “scaffold” means a 3-dimensional structure which can support cell cultures, including primary cells, immortalized cells, stem cells in the form of sparse cells or organoids, spheroids and tissue explants as well as microfluidic perfusion of liquids including cell culture media, buffers and blood.

While the following terms are believed to be well understood by one of ordinary skill in the art, definitions are set forth to facilitate explanation of the disclosed subject matter. Unless defined otherwise, all technical and scientific terms used herein have the same meaning as commonly understood by one of ordinary skill in the art to which the disclosed subject matter belongs.

### DETAILED DESCRIPTION OF THE INVENTION

One or more specific embodiments of the present invention will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

One skilled in the art will recognize that the various embodiments may be practiced without one or more of the specific details described herein, or with other replacement and/or additional methods, materials, or components. In other instances, well-known structures, materials, or operations are not shown or described in detail herein to avoid obscuring aspects of various embodiments of the invention. Similarly, for purposes of explanation, specific numbers, materials, and configurations are set forth herein in order to provide a thorough understanding of the invention. Furthermore, it is understood that the various embodiments shown in the figures are illustrative representations and are not necessarily drawn to scale.

Reference throughout this specification to “one embodiment” or “an embodiment” means that a particular feature, structure, material, or characteristic described in connection with the embodiment is included in at least one embodiment of the invention but does not denote that they are present in every embodiment. Thus, the appearances of the phrases “in an embodiment” or “in another embodiment” in various places throughout this specification are not necessarily referring to the same embodiment of the invention. Further, “a component” may be representative of one or more components and, thus, may be used herein to mean “at least one.”

The present invention concerns a new type of vascular tissue model. In one embodiment, the model uses a microfluidic scaffold with two or more inlets at the top of the device and a central chamber. The microfluidic scaffold is created using an industrial three-dimensional (3D) printing technique such as stereolithography (SLA). The central chamber contains a hydrogel lined with a microfluidic channel. The hydrogel is created using 3D bioprinting using a sacrificial bio-ink. The microfluidic channel connects the hydrogel to the scaffold. The hydrogel typically includes stromal cells or brain glioma cells. In one embodiment, the hydrogel is made of materials such as fibrin, collagen, Matrigel, alginate, gelatin, synthetic biopolymers such as PEG or tissue-specific extracellular matrix obtained from human donors or animals. These materials allow the hydrogel to faithfully replicate the natural 3D microenvironment of a living tissue. In one embodiment of the invention, the vascular tissue model is of the human blood-brain barrier (BBB) and can demonstrate an in-vitro system of glioblastoma. In this embodiment, the microfluidic channel contains human endothelial cells.

Importantly, by not using PDMS or membranes, the system of the present invention has the advantages of scalability and rapid manufacturing. Instead of taking weeks, many systems can be created in a day. The system of the present invention is more cost-effective. The system more accurately replicates organ systems and can be applied to more types of vascularized systems.

Despite the tremendous research efforts made by scientists, neurological diseases remain the main cause of disability and death worldwide. Neuronal cell death and degeneration have been the center of extensive research work in the field of neurological diseases; however, recent experimental shreds of evidence have led to a paradigm shift and more attention is being paid to the role of the brain vasculature. Defects in the blood-brain barrier (BBB) are associated with a variety of brain diseases, including brain cancer, stroke, and neurodegenerative conditions. Given the obvious discrepancies across species, manifested by the poor predictive power of animal models for human responses to drugs, the relevance of animal models to study neurological diseases is questionable. While conventional in vitro models are simply not conceived neither for capturing the tridimensional tissue architecture nor for recapitulating the highly dynamic events occurring at the BBB, microphysiological systems (MPSs) have recently emerged as an alternative solution. The present invention is a next-generation BBB-MPS obtained from the combined use of industrial 3D- and bio-printing methods to generate a robust and scalable model of the human BBB. This inventive system recapitulates the endothelial-parenchymal interface of the BBB, maintains high barrier function, and allows the simulation of dynamic fluid flow for the study of brain diseases such as glioblastoma. In a dynamic co-culture system of human endothelial and brain cancer cells in the bio-printable hydrogel, the system demonstrates fluid shear regulated phenotypical change of cancer cells, indicating the role of shear stimulus for glioblastoma heterogeneity. These results support the use of the BBB-MPS for a wide range of studies aimed at detecting potential drug toxicity and efficacy, as well as the basic to complex processes underlying brain homeostasis and inflammation.

### Limitations of Previous MPSs

Microfluidic models of the human BBB have been developed, comprising human astrocytes, pericytes and microvascular endothelial cells, capable of mimicking the selective barrier-function of the BBB. These microphysiological systems (MPSs) have been shown to respond to various inflammatory cues and to sustain human blood flow, providing an improved platform for modeling of blood-endothelial cell interactions. However, current microfabrication methods and chip designs are major limiting factors in modeling the 3D tissue architecture and function of the human NVU. Microfabrication of MPSs has typically been based on the concept of creating microchannels where cells grow in a designed space, defined by synthetic chemical materials that cells cannot remodel. Previous MPSs of the BBB have been built using polydimethylsiloxane (PDMS), which functions as the designed space, on parallel microfluidic chambers separated by a porous membrane. These MSP designs suffer from multiple drawbacks. (A) PDMS properties result in absorption of small molecules, including neurotransmitters and therapeutics. Therefore, current MPS designs cannot be used for drug testing and assessment of soluble cell metabolites. (B) Use of a membrane results in asymmetrical formation of the tissue-tissue interface, confined to only one specific surface of the vascular channel. As a result, these designs inhibit the natural ability of cells to remodel the surrounding microenvironment and to reposition following biochemical signals, with consequent limited formation of intercellular interactions. (C) Typical microfabrication methods require the use of expensive facilities and long prototyping steps.

### PDMS Absorbs Small Molecules

Small molecules are currently the largest class of therapeutics targeting the central nervous system. However, there is some difficulty in developing small molecules for the treatment of CNS diseases due to lack of efficacy (accounting for some 30% of attrition) and toxicity (accounting for some 30% of drug failures). Though preclinical animal models reproduce some physiological aspects of human disease, they lack the complexity of the human brain and hence do not predict human response in terms of efficacy and toxicity. MPSs offer the opportunity to replicate the structure and function of human tissues in a mechanically active and controlled microenvironment. Polydimethylsiloxane (PDMS) is the most widely used material for fabrication of MPSs because it is inexpensive and has prototyping-friendly properties. However, PDMS exhibits variable and time-dependent absorption of small, hydrophobic molecules, making it unsuitable for drug testing and assessment of soluble cell metabolites (<900 Da), including most neurotransmitters. An MPS that is compatible with testing of novel small molecules drug-candidates could significantly enhance the development of novel therapeutics as well as research to elucidate the mechanisms of drug-induced toxicity.

Instead of PDMS, an embodiment of the present invention employs a hydrogel that provides cells with a highly biocompatible and hydrophilic microenvironment and does not absorb small molecules.

### Asymmetrical Designs

Most MPSs still rely on fabrication methods that use a patterned layer of PDMS to fabricate microfluidic parallel chambers separated by a porous membrane, sometimes referred to as a “sandwich” configuration. In these devices, only one surface (frequently the upper) of the vascular chamber can interface with other cell types (such as astrocytes), creating a non-physiological and asymmetric tissue structure. Only a fraction of the vascular surface is accessible to the astrocytes when these cells are co-cultured in a conventional sandwich-type device. The device of the present invention, on the other hand, allows for co-culturing of human induced pluripotent stem cell (iPSc)-derived astrocytes (iAstrocytes) and brain microvascualr endothelial cells (iHBMECs) for microengineering of an iPSc-derived BBB-on-Chip.

When in co-culture with iHBMECs, iAstrocytes are capable of self-arranging end-feet like structure protruding through the porous membrane and connecting the neuronal chamber with the vascular chamber. However, we found that less than 1% of the vascular surface was covered with astrocytic end-feet like structures, which is far from the physiological end-feet/vascular coverage estimated in a healthy brain tissue ranging from 80% to 99%. Similar results were reported by another group for a different chip design that also employed the parallel microfluidic chamber approach for culturing human astrocytes (embedded in a hydrogel) next to brain endothelial cells. These observations indicate that a porous membrane separating the two cell types physically impedes the astrocytes' intrinsic ability to sense and connect with the vascular endothelium. A different approach that does not employ a membrane is needed to better model the tissue organization of the human CNS.

Results obtained through computer modelling of flow dynamics into a microfluidic chamber revealed that a microchannel of circular cross section obtained through bioprinting methods, differently than rectangular microchambers made of PDMS, provide a more physiologically relevant geometry capable of re-distributing the shear force homogeneously, as it naturally occurs in our body. These results indicate that round microchannels obtained via bio-printing of sacrificial materials can be used for recapitulating the anatomical shape and physiological blood-flow dynamic of blood-vessels, and therefore represent a better alternative to rectangular chambers used in traditional PDSM-made devices.

### The Emergence of Bioprinted Microphysiological Systems

In the past 20 years, the combined use of soft lithography (Table 1), photolithography and PDMS resulted in an enabling microfabrication strategy that allowed a growing number of researchers to generate, in a relative short time-period, a variety of OOC models with demonstrated tissue and organ-level function. Under the increasing demand for more complex and predictive in vitro models for drug testing, the field of OOCs has rapidly evolved to incorporating multiple tissue-specific cells including stromal, microvascular endothelial and immune cells. Despite the different fabrication strategies, the use of parallel microfluidic chambers, frequently made of PDSM, remains the most prominent design feature across nearly all existing OOCs and MPSs. Currently, a shift in the paradigm is in progress as some of the most recent publications suggest that the field is moving away from the concept of confining cells in parallel microfluidic chambers made of PDMS (or other plastics) as hydrogels and other bio-inspired materials are taking the place of traditional synthetic polymers, a transition often enabled by 3D printing.

TABLE 1

| Fabrication Method | Advantage | Disadvantage |
|---|---|---|
| Soft Lithography | Time saving, inexpensive, | Structure needs to be continuous, |
| | reusable molds from single | other technique such as |
| | master, easier setup, | photolithography, |
| | high throughput, wide range of | e beam is required to fabricate |
| | resolution | master stamp once |
| Photolithography | Robust, repeatable, resolution | Requires expensive equipment, |
| | ranges from micron to | and specialized training, time |
| | millimeter | consuming, limited to |
| | | photocrosslinkable materials |
| Etching | High resolution, sub | Chemicals(acid/base) may damage |
| | micrometer size, well known | the polymers, Poor process control |
| | technique | and selectivity due to temperature |
| | | gradient, multiple steps, high cost |
| | | and time |
| Laser cutting | Robust, time saving, allows for | Laser cutting of plastic |
| | alternative plastics that replace | components can be expensive as it |
| | PDMS | requires the use of ventilated |
| | | rooms or instruments designed to |
| | | remove harmful gas |

At the beginning of the 21st century, bioprinters were only accessible to a limited number of researchers who worked in specialized bioengineering laboratories. Since then, through the diffusion of commercial bioprinters, a new wave of scientists gained access to the bioprinting technology. Researchers have tackled increasingly difficult problems, including the ability to vascularize thick micro-tissues obtained from the 3D bioprinting of multiple layers of cell-laden bioink. Bioprinting supports specific functionalities and biomaterial heterogeneity which could mimic the functionality of cells. This enables the creation of a biomimetic microenvironment with heterogeneous 3D structures that cannot be obtained with traditional microfabrication methods.

### Design of the Present Invention

The present invention enables the development of Microphysiological in vitro models of vascularized human tissues for (i) pre-clinical testing of novel drug candidates, (ii) repurposing of existing drugs, and (iii) developing of personalized in vitro models using patient-derived cells. The systems are designed to recapitulate specific architecture and biomechanical cues that are central to modelling structure and function of vascularized human tissues. As opposed to existing MPSs and Organ-on-Chip models, the systems of the present invention are microfabricated via a combination or 3D printing and Bioprinting. 3D printing is used to generate a rigid scaffold lined with a microfluidic channel and designed to host a perfusable cell-laden hydrogel obtained via bio-printing of sacrificial material.

The terms “Microphysiological system” and “Organ-on-Chip” are frequently used as synonyms, both referring to microfluidic-based in vitro models designed to capture the dynamic biochemical microenvironment of living organs. Organ-on-Chip are “called ‘chips’ because they were originally fabricated using methods adapted from those used for manufacturing of computer microchips” such as soft-lithography. The approach of the present invention does not conceive the use of any fabrication method that is related to manufacturing of electronic chips. However, we will use the word “Chip” to refer to the microfluidic chamber used as a rigid microfluidic scaffold.

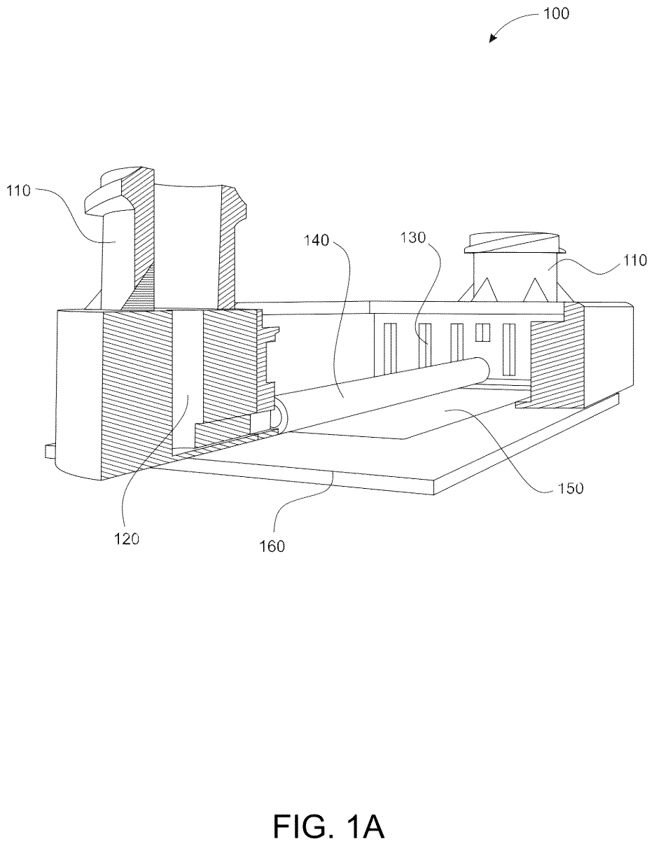

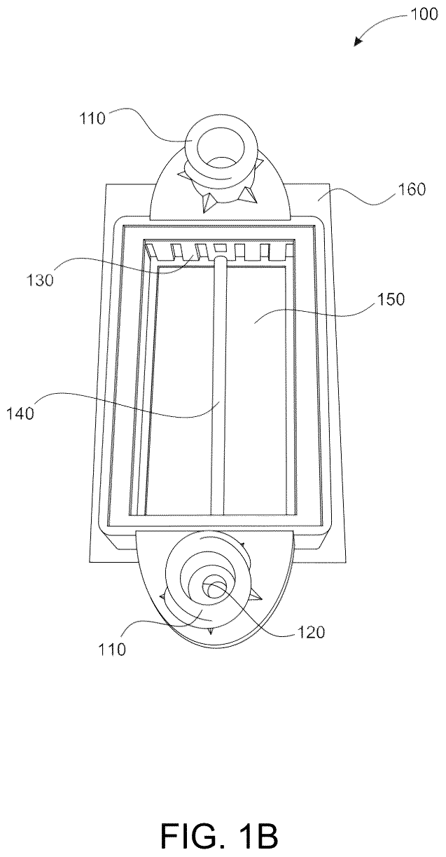

Our innovative approach allows for integrating three-dimensional (3D) cell culture methods with microfluidic principles to generate a perfusable 3D microtissue. In one embodiment, the system consists of one 3D printed microfluidic scaffold obtained via stereolithography (SLA) and used as frame to host a bio-printable cell-laden hydrogel. Referring to FIGS. 1A and 1B, one or more microfluidic channels connect the rigid scaffold with the hydrogel making this a whole perfusable unit. The 3D printed scaffold 100 incorporates two or more microfluidic ports 110 and inlets 120 designed to facilitate the connection to commercially available pumps. For example, the microfluidic ports 110 located at the top of the device facilitate microfluidic perfusion using commercially available luer-lock fittings. The inner surface of the 3D printed scaffold is lined with hydrogel anchoring structures 130 (or grooves) designed to enable long term (e.g., >10 days) cell culture under fluid flow. The hydrogel can be prepared to encapsulate living cells to mimic the stromal component of living organs. Sacrificial bioinks (such as pluronic or gelatin) may be used to bio-print a perfusive microchannel inside the hydrogel. Importantly, the hydrogel can be made of different biocompatible materials such as fibrin, collagen, Matrigel, alginate, gelatin or synthetic biopolymers such as PEG or tissue-specific extracellular matrix obtained from human donors or animals in order to faithfully reconstitute the natural 3D microenvironment of a living tissue. The fluidic microchannel 140 embedded into the hydrogel can be seeded with endothelial cells. Before cell seeding, the hollow surface of the hydrogel is coated with tissue-specific extracellular matrix proteins (such as collagen IV) to better reflect the tissue composition of the blood vessels. Ultimately, the use of a 3D hydrogel 150 enables the bio-fabrication of a vascularized synthetic microtissue comprising a perfusable endothelial microchannel surrounded by tissue-specific stromal cells, such as brain astrocytes. The bottom surface of the device 160 is made of transparent glass or vinyl both compatible with conventional microscopes and other optic systems (such as plate readers). The chip's transparency allows researchers to see the organ's functionality, behavior, and response, at the cellular and molecular level.

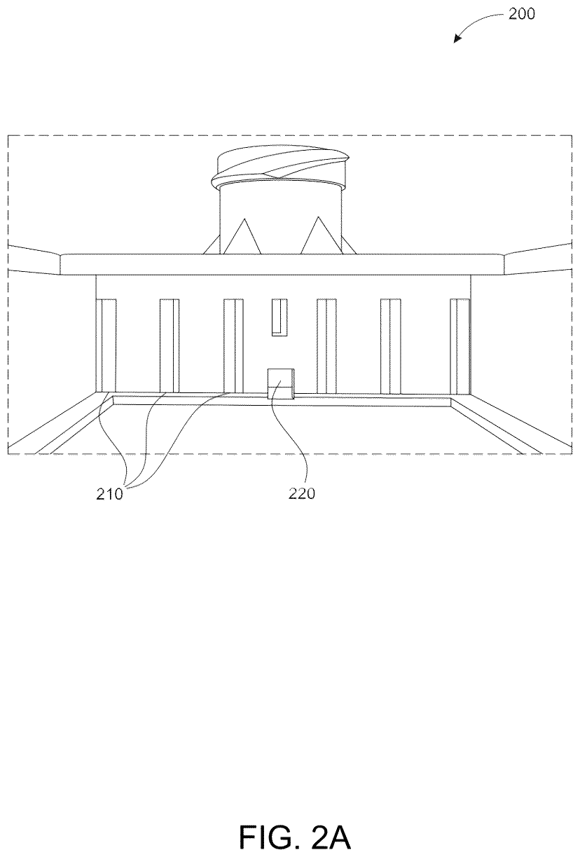

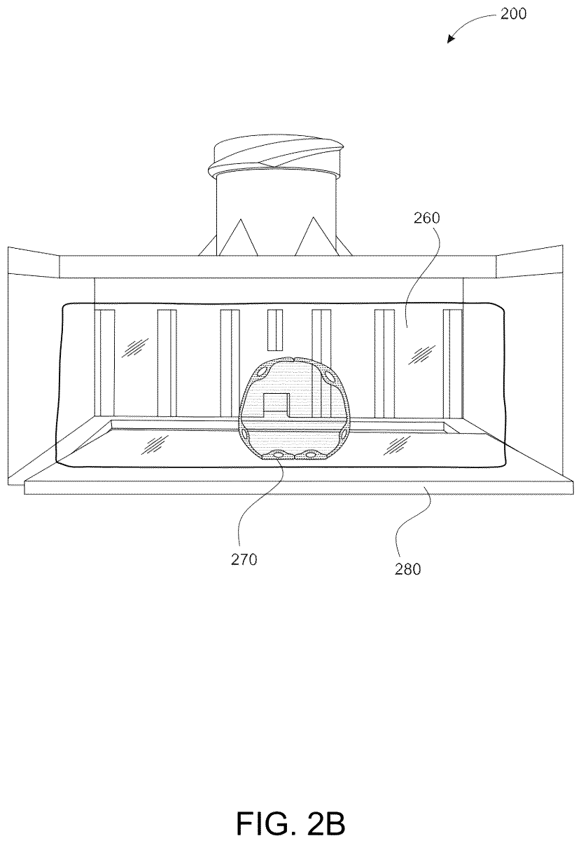

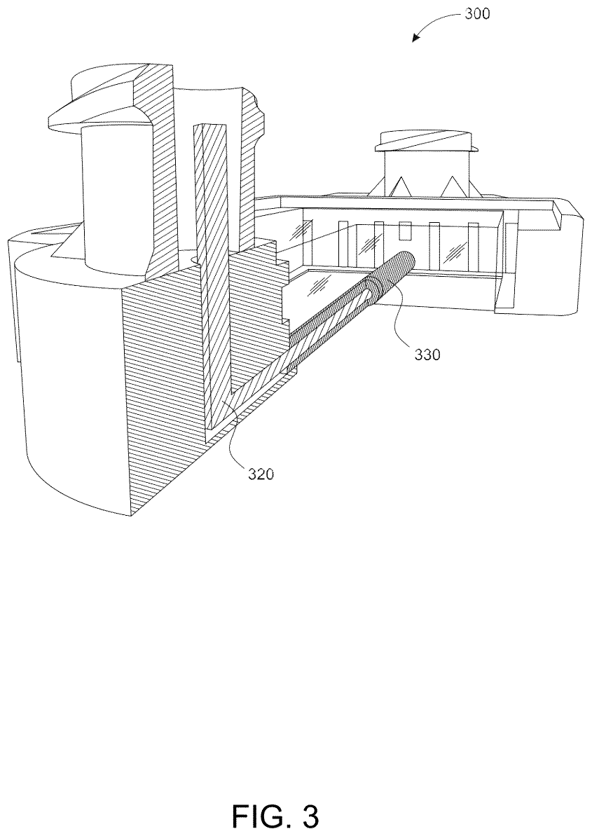

FIGS. 2A and 2B provide an alternate view of the central chamber of the device 200, which is designed to host a perfusable hydrogel 260 lined with a hollow microchannel obtained via bio-printing of sacrificial bio-ink. In one embodiment, the hydrogel includes stromal cells such as astrocytes or brain glioma cells. Human endothelial cells may be seeded in the microfluidic channel one or two days after gel polymerization to form a vascular channel. The interior of the device includes grooves 210 to help anchor hydrogel and a microfluidic inlet 220 (see FIG. 2A). A hydrogel 260 is placed on a transparent glass slide 280 (see FIG. 2B). A vascular channel 270 runs through the hydrogel 260. FIG. 3 shows that when the device 300 is in use, fluid flow 320 passes inside a vascular wall 330.

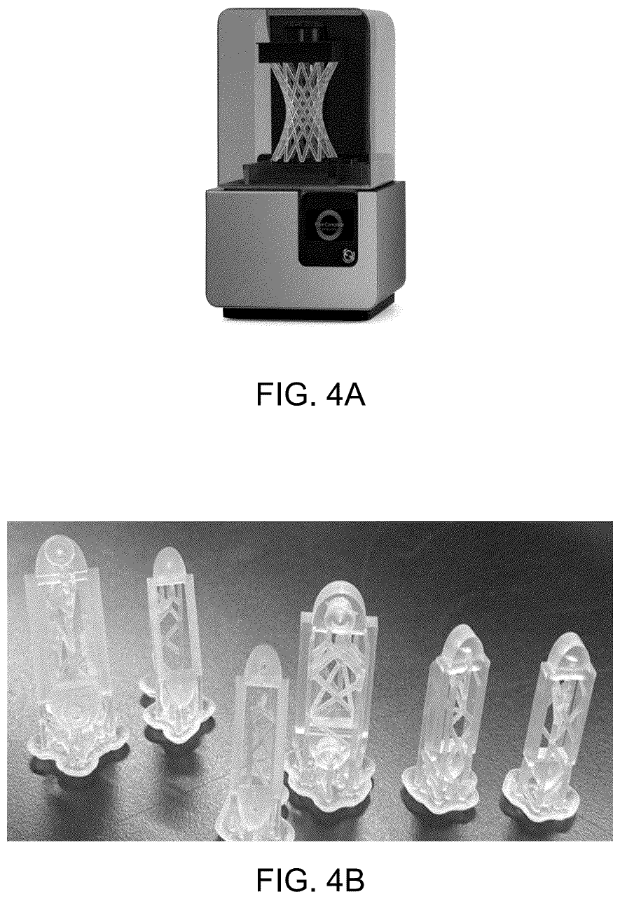

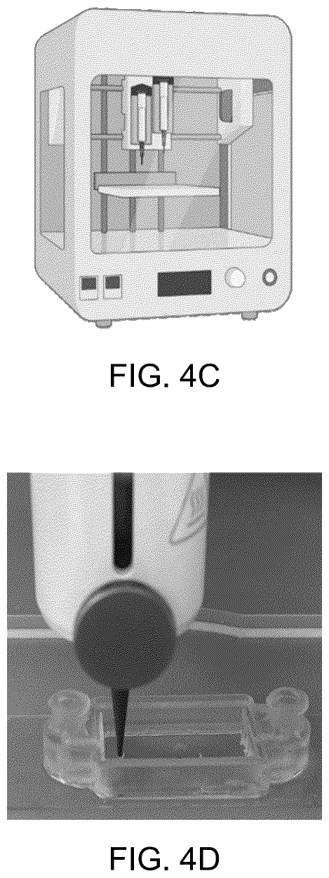

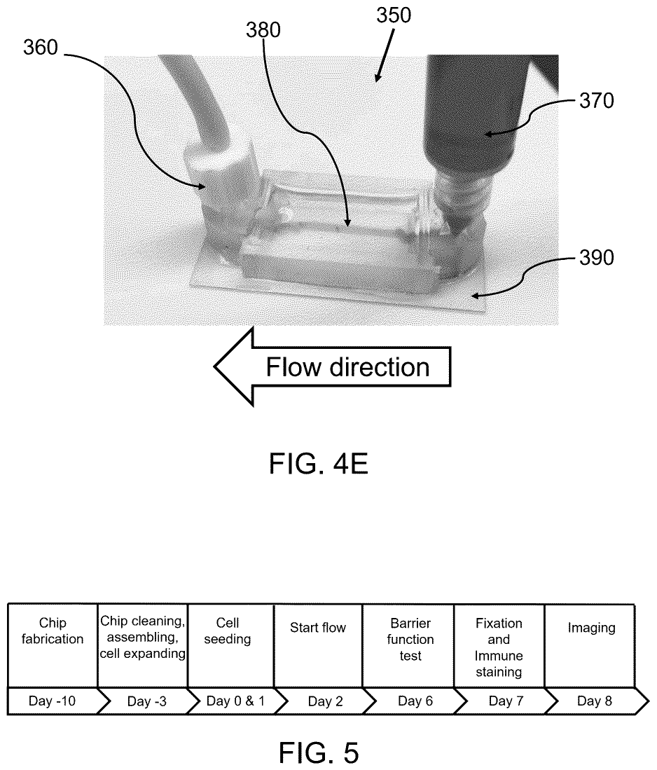

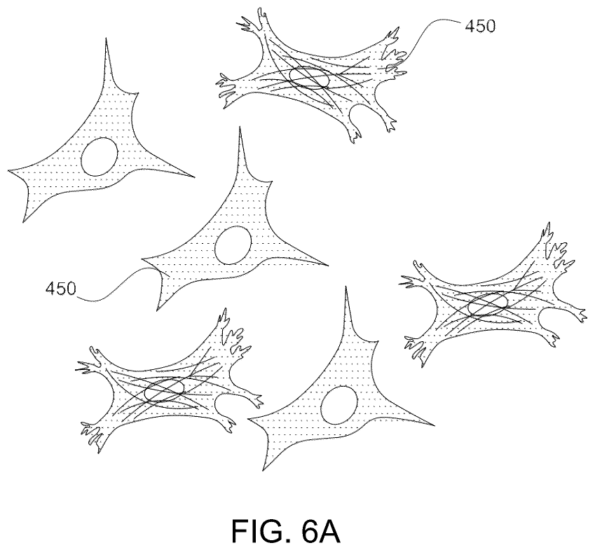

The combined use of 3D printing and bioprinting represents an enabling approach toward generating scalable and complex vascularized microphysiological systems. Referring to FIG. 4A, an industrial 3D printer (SLA) is used to produce a microfluidic scaffold. FIG. 4B shows a number of printed scaffolds. FIG. 4C shows an image of a 3D bioprinter used to bioprint a microfluidic vascular channel. FIG. 4D is an image of the bioprinter printing such a channel. The system of the present invention can be connected with commercially available microfluidic fittings to generate physiologically relevant vascular shear stress. FIG. 4E is a photo of a system 350 with an outlet 360, a vascularized channel 380, and a transparent surface 390. Medium 370 flows through the vascularized channel 380 in the flow direction indicated in the figure. The system of the present invention can be further adapted to produce conventional analytical readouts for detecting/analyzing, among other things, cell viability, vascular barrier function, immune staining, fluorescence microscopy and metabolic assays. FIG. 5 is a schematic showing the key steps of one embodiment of the approach of the present invention.

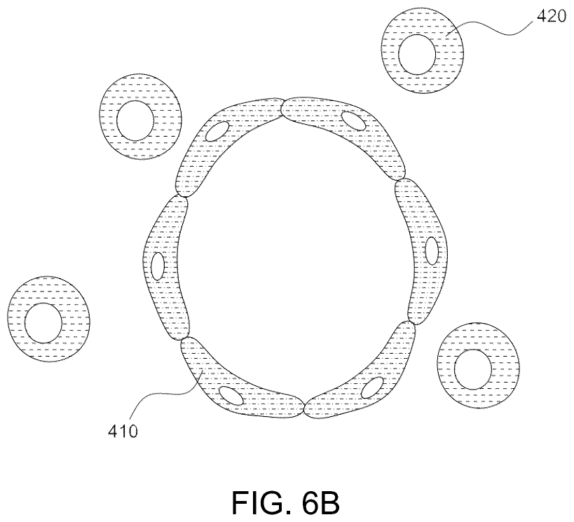

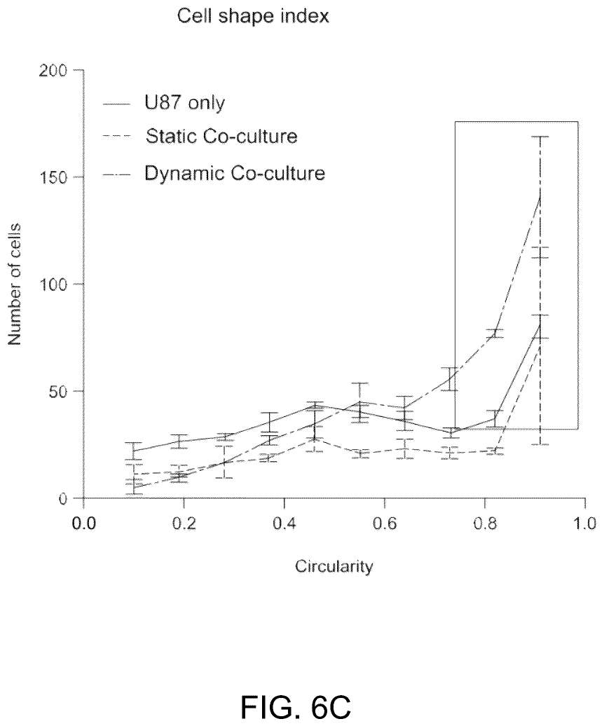

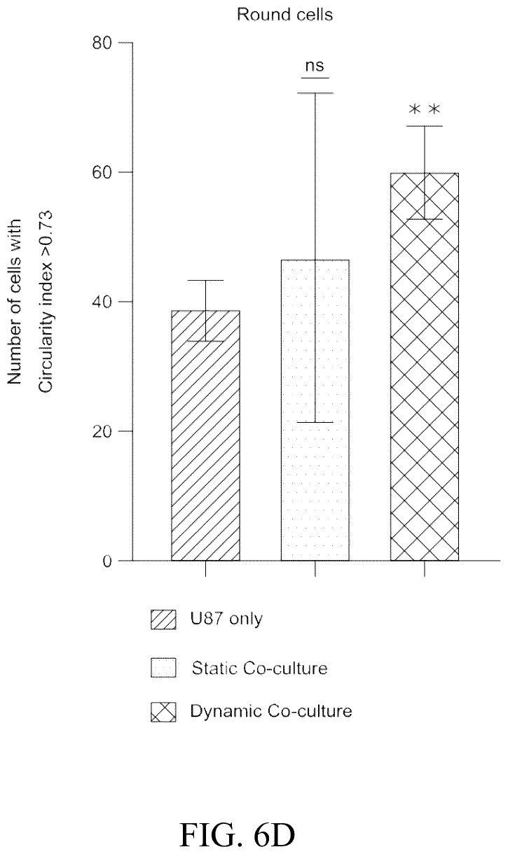

The device of the present invention can be used with monocultures or co-cultures in static or dynamic systems. It was found that the shear in a dynamic co-culture system induces a morphological change of glioblastoma. More specifically, it was discovered that cells contained in the hydrogel can communicate with each other and that the presence of fluid-flow combined with the co-culture of heathy endothelial cells and brain cancer cells result in “differentiation” of cancer cells into two subpopulation with clear morphological differences. The cells near the endothelium appear round and express CD133, a marker specifically found on cancer stem cells. Cells far from the endothelium appear branched and are negative for CD133. Morphology (roundness) can be measured and the system can also be used to assess the presence of pro-inflammatory mediators (cytokines) and other soluble biomarkers and other factors released by cells in culture. Referring the FIGS. 6A and 6B, cancer cells show a branched morphology 450 in monoculture and static co-culture and a roundish morphology 420 near endothelial cells 410 in dynamic co-culture. FIGS. 6C and 6D are graphs showing the results of morphological change in cells. The highest number of cells with roundish phenotype is observed in a dynamic co-culture system.

### Method of the Present Invention

It is challenging to build a bio-compatible and air-tight microfluidic system. 3D printing via SLA allows for rapid manufacturing of 3D objects including microfluidic channels, however, the material used in SLA printing (often named resins) are usually not transparent and not biocompatible. Further, selecting a glue to bond a glass slide to the 3D printed part is not trivial. Resins tend to impair the polymerization of silicon (and other polymers), as previously observed by others.

For the present invention, it was discovered that both the intrinsic cytotoxicity and the anti-polymerization properties of the 3D printed parts were quenched after overnight incubation in ethanol or isopropanol. A concentration of at least 75% was used. In one embodiment, the concentration of ethanol or isopropanol used for incubation is at least 90%. A combination of epoxy, silicones, or UV-curing resin (sometimes named “liquid plastic”) was used to bond and seal 3D printed parts to transparent surfaces, either glass or vinyl. In one embodiment, a rinsing step is used to ensure full biocompatibility with human cells. Further, in one embodiment, the present invention uses surface functionalization to achieve long-term cell culture in these devices. A chemical silanizing agent (APTES) was used to modify the surface of the device and allow for covalent bonding of the hydrogel to the inner surface of the device.

### GBM-On-Chip

Conventional 2D cell cultures and 3D spheroid have been used to assess the effects of drugs on tumor cell growth for over 50 and 30 years, respectively. However, they do not provide information about the complex interactions between the cancer cells, associated stromal components and the dynamic microenvironment that exists within the tumoral mass that form within the brain tissue. The emerging organ-on-chip technology offers unique opportunities for dissecting the intercellular communication occurring within GBM and surrounding microenvironment and testing of drug compounds for the purpose of developing novel and personalized therapeutic strategies. Unfortunately, previous models suffered from a few drawbacks associated with conventional microfabrication methods and materials that represents a major obstacle to the development of a predictive GBM-on-chip model suitable for drug testing. PDMS is the most widely used material for fabrication of organ-on-chip because it is inexpensive and has prototyping-friendly properties. However, PDMS exhibits variable and time-dependent absorption of small, hydrophobic molecules, making it possibly unsuitable for drug testing of small molecules, which account for the largest class of therapeutics targeting the central nervous system. An organ-on-chip model of GBM that is compatible with testing of small molecule drug-candidates could significantly enhance the development of novel therapeutics as well as research to elucidate the mechanisms of brain cancer progression and drug resistance.

The present invention is an organ-on-chip model designed to mimic the multicellular architecture of the BBB compatible with testing of small molecule compounds. The system is designed to closely resemble the anatomical 3D structure of the BBB, composed by a vascularized microfluidic compartment lined with endothelial cells directly interfaced with astrocytes and neurons harbored in a full 3D hydrogel. Unlike PDMS, the hydrogel provides cells with a natural 3D space where cells can remodel the surrounding environment and physically migrate and reposition in all the three spatial dimensions to assume their native configuration and recreate higher order tissue-structures such as astrocytic end-feet. When compared to previous “sandwich” chip designs where cells were grown on flat surfaces, the present 3D printing approach enables the generation of a full-3D environment that maximizes the surface available for tissue-tissue interactions and provides homogeneous exposure to the cell factors and direct cancer-stroma interactions implied in GBM disease progression.

The GBM-on-chip of the present invention is designed to capture the complex intercellular interplay that occurs at the BBB between cancer cells and the stroma, which is central to gaining new insights into the role of the tumor microenvironment in cancer resistance and the identification of novel therapeutic strategies. The GBM-on-chip design of the present invention is novel in that, in one embodiment, it is bioprinted and stem cell derived, offering the potential to: accurately represent the complex 3-dimensional structure of the BBB and analyze the formation of intercellular interactions; test novel, small-molecule drug candidates and model cancer cell selection to chemotherapy, including clonal selection and BTB mediated resistance. This technology affords the ability to scale the number of treatment strategies tested, allows monitoring of the drug effects on normal tissues in addition to the anticancer effects, and supports the development of novel therapies against microenvironmental targets.

One novel aspect of the GBM-on-chip design is that it combines 3D-printing of rigid materials with bioprinted microfluidic hydrogels. Additionally, it includes elements of both the hypoxic and perivascular niches of GBM. When compared to previous microfluidic models where cells are grown onto flat surfaces or rigid parallel chambers made of silicon (PDMS), the 3D bioprinted design of the present invention (a) enables microfabrication of a dynamic 3D environment; (b) maximizes the surface available for tissue-tissue interactions while providing a physiologically-relevant substrate that cells can remodel; and (c) promote direct cancer-stromal cell interactions that are critical for modeling the dynamic progression of brain cancer. This technology has the potential to provide a new way to recapitulate intra-tumoral heterogeneity and can be deployed for drug screening.

In addition to cancer cells, the present invention can be used to study cancer spheroids and organoids. Interestingly, when cancer spheroids are introduced in the system and co-cultured with endothelial cells and in presence of vascular flow, cancer spheroids are more resilient to drug (TMZ) treatment when compared to spheroids cultured in conventional conditions: without endothelial cells and without flow.

All documents cited are incorporated herein by reference; the citation of any document is not to be construed as an admission that it is prior art with respect to the present invention.

It is to be further understood that where descriptions of various embodiments use the term “comprising,” and/or “including” those skilled in the art would understand that in some specific instances, an embodiment can be alternatively described using language “consisting essentially of” or “consisting of.”

While particular embodiments of the present invention have been illustrated and described, it would be obvious to one skilled in the art that various other changes and modifications can be made without departing from the spirit and scope of the invention. It is therefore intended to cover in the appended claims all such changes and modifications that are within the scope of this invention.

What is claimed is:

1. A vascular tissue model comprising a rigid 3D printed scaffold, the scaffold comprising:
a. one or more scaffold microfluidic channels;
b. two or more inlets; and
c. a central chamber,
wherein the central chamber contains a hydrogel, wherein the hydrogel contains a hydrogel microfluidic channel.

2. The vascular tissue model of claim 1 wherein the hydrogel microfluidic channel connects to one or more of the scaffold microfluidic channels.

3. The vascular tissue model of claim 1 wherein the inlets are capable of connecting to one or more pumps.

4. The vascular tissue model of claim 1, wherein the scaffold has an inner surface and the inner surface comprises one or more hydrogel anchoring structures.

5. The vascular tissue model of claim 1 wherein the rigid 3D printed scaffold is created using stereolithography.

6. The vascular tissue model of claim 1 wherein the microfluidic scaffold comprises a transparent resin, and further, wherein the microfluidic scaffold is biocompatible with biological material that may be used in the vascular tissue model.

7. The vascular tissue model of claim 1 wherein the microfluidic scaffold is surface functionalized.

8. The vascular tissue model of claim 1 wherein the hydrogel is created using three-dimensional bioprinting.

9. The vascular tissue model of claim 1 wherein the hydrogel microfluidic channel has a circular cross section.

10. The vascular tissue model of claim 1 wherein the hydrogel comprises a material selected from the group consisting of fibrin, collagen, matrigel, alginate, gelatin, synthetic polymers, and tissue-specific extracellular matrix.

11. The vascular tissue model of claim 1 wherein the hydrogel comprises stromal cells, brain glioma cells or combinations thereof.

12. The vascular tissue model of claim 1 wherein the hydrogel microfluidic channel contains human endothelial cells.

13. The vascular tissue model of claim 1 wherein the vascular tissue model is of the human blood-brain barrier.

14. A method of modeling a vascular tissue system comprising:
a. inserting a culture comprising cancer cells in the hydrogel microfluidic channel of the vascular tissue model of claim 1;
b. connecting the vascular tissue model to one or more pumps;
c. flowing medium through the vascular tissue model, including the hydrogel microfluidic channel; and
d. collecting data regarding the culture in the hydrogel microfluidic channel.

15. The method of claim 14 wherein the rigid 3D printed scaffold is created using stereolithography.

16. The method of claim 14 wherein the hydrogel is created using three-dimensional bioprinting.

17. The method of claim 14 wherein the culture is a co-culture of human endothelial cells with cancer cells.

18. The method of claim 14 wherein the cancer cells are brain glioma cells.

19. The method of claim 14 wherein the culture comprises stromal cells and brain glioma cells.

20. The method of claim 19 wherein the stromal cells comprise endothelial cells, astroglia cells or combinations thereof.
